(19) United States
(12) Patent Application Publication
(10) Pub. No.: US 2008/0032251 A1
(43) Pub. Date: Feb. 7, 2008

(54) Laser carious region ablation

(76) Inventor: Mau-Song Chou, Rancho Palos Verdes, CA (US)

Correspondence Address: PATTI, HEWITT & AREZINA LLC, ONE NORTH LASALLE STREET, 44TH FLOOR, CHICAGO, IL, 60602, US

(21) Appl. No.: 11/489,055

(22) Filed: Jul. 19, 2006

(51) Int. Cl. A61C 3/00; A61B 18/18

(52) U.S. Cl. 433/29; 606/10

(57) ABSTRACT

A coolant delivery system of an apparatus in one example creates a layer of coolant over a target area. The target area comprises at least one carious region. A laser generator of the apparatus directs a laser beam to the target area to ablate the at least one carious region. The laser generator operates substantially simultaneously with the coolant delivery system to direct the laser beam through the layer of coolant to the target area. The layer of coolant cools the target area to reduce thermal damage from the laser beam.

### TECHNICAL FIELD

The invention relates generally to dentistry and more particularly to laser dentistry.

### SUMMARY

The invention in one implementation encompasses an apparatus. The apparatus comprises a coolant delivery system that creates a layer of coolant over a target area. The target area comprises at least one carious region. A laser generator directs a laser beam to the target area to ablate the at least one carious region. The laser generator operates substantially simultaneously with the coolant delivery system to direct the laser beam through the layer of coolant to the target area. The layer of coolant cools the target area to reduce thermal damage from the laser beam.

Another implementation of the invention encompasses an apparatus. The apparatus comprises a handpiece, a coolant delivery system, and a laser generator. The handpiece is coupled with the coolant delivery system to receive a supply of coolant. The handpiece comprises an optical fiber that is optically coupled with the laser generator to receive a laser beam from the laser generator. The handpiece is configured to create a layer of coolant over a target area, wherein the target area comprises at least one carious region. The handpiece is configured to direct the laser beam through the layer of coolant to the target area to ablate the at least one carious region. The handpiece substantially simultaneously creates the layer of coolant over the target area and directs the laser beam through the layer of coolant to the target area to ablate the at least one carious region. The layer of coolant cools the target area to reduce thermal damage from the laser beam.

A further implementation of the invention encompasses a method. A pool of coolant over a target area is created for cooling of the target area. The target area comprises at least one carious region. An infrared laser beam is substantially simultaneously directed through the pool of coolant to the at least one carious region to ablate the at least one carious region.

### DESCRIPTION OF THE DRAWINGS

FIG. 1 is a schematic view of one implementation of an apparatus comprising a laser system, a coolant system, and a suction system.

FIG. 2 is another schematic view of the apparatus of FIG. 1.

FIG. 3 is a schematic view of the coolant delivery system of FIG. 1.

FIG. 4 is a schematic view of the suction system of FIG. 1.

FIG. 5 is a schematic view of an alternative implementation of the laser system and coolant system of the apparatus of FIG. 1.

### DETAILED DESCRIPTION

As is generally known in the art of dentistry, conventional drilling machines for treating carious regions such as plaque and decay tissue can be inaccurate and painful. Therefore, it is desirable to replace or support conventional drilling machine with lasers in order to achieve a more accurate and painless treatment of carious regions. However, laser irradiation processes currently available often produce charring on the target surface and surrounding areas due to laser generated heat. The blackened char tissue effectively blocks the laser radiation thereby preventing it from reaching biological tissue thereunder. Thus, charring due to excess heat interrupts the ablation process. In addition, excess heat may also produce cracks in the tooth surface and damage the nerve system and other pulp structure in the pulp chamber irreversibly. Therefore, it is desirable to provide an apparatus and method for treating carious regions using laser irradiation which does not generate sufficient heat to cause thermal damage.

Vassiliadis, et al. (U.S. Pat. No. 5,180,304) disclose a dental cutting method by spraying water on a tooth following each laser pulse. The '304 patent stresses that the tooth needs to be dried prior to a subsequent laser pulse in order to minimize attenuation of the laser radiation by the water. A drying means, such as an air sprayer is generally used to dry the tooth surface before a subsequent laser pulse is applied. The patent may not provide adequate cutting speeded for dental applications since a drying step before application of subsequent laser pulse will slow down a treatment process.

Steiner et al. (U.S. Pat. No. 5,199,870) disclose a dental cutting method that energizes a film of water using laser radiation. The expansion of water provides means to cause the destruction and removal of tooth material. The process requires a critical control of the thickness of the water film for laser-induced expansion of the water film.

Wolbarsht, et al. (U.S. Pat. No. 5,267,856) disclose a dental cutting method by use of a thin layer of water that enters the surface pores or is chemically held on the surface of the tooth. The '856 patent stresses that the water is not permitted to remain pooled on the tooth surface. Since dental material are not very porous, there is a high degree of difficulty to insert water into the pores.

Rizoiu et al. (U.S. Pat. Nos. 5,741,247, 5,785,521, 5,968,037, 6,254,597 B1, 6,231,567,B1, 6,350,123, B1, 6,610,053 B1, 6,669,685 B1) disclose a method of dental cutting by use of an atomizer to produce a mist of fine water droplets. The fine water droplets are injected into a volume of air adjacent to the target surface. An electromagnetic energy source focuses electromagnetic energy into the fine water droplets in the volume of air. As the water droplets are heated by the laser radiation, the droplets expanded and exploded. Explosion of the water droplets imparts mechanical cutting into the target surface to provide a means of cutting the target surface. Rizoiu et al. patents stress that a layer of water preferably does not cover the target surface. Rizoiu et al. patents have met some success, however, it is difficult to maintain and confine the fine water droplets in the volume of air in front of a target without forming a layer of water that can cover the surface of the target.

Neuberger (U.S. Pat. No. 6,758,844 B2) discloses a method of treating oral tissues by use of a radiation source emitting at a wavelength of about 980 nm and a liquid/gas spray onto the treatment area. The liquid/gas spray flushes away tissue debris in addition to cooling the treated tissue. The '844 patent claimed the use of 980-nm radiation for treatment. However, the 980 nm laser may not provide an optimal wavelength for cutting of hard dental tissue.

LaBudde et al. (U.S. Pat. No. 6,607,524) disclose a dental cutting method by intermittently applying an electromagnetic energy and water alternately on to a target surface. Electromagnetic energy is applied to a target material to result in fracture and rupture of the material. The energy source is stopped and immediately a fluid is applied to the material with sufficient cooling power when the material reaches to the point of fracture and rupture. The process is repeated until the desired cutting is affected. The use of water spray intermittently may not be a time-efficient process for cutting material.

U.S. Pat. No. 6,083,218, issued to the applicant on Jul. 4, 2000, discloses an optical method for removing tooth material by use of a water coolant delivery system and an ultraviolet laser system. The water coolant has an arbitrary thickness on the target surface, removes excess heat, and prevents charring the surface tissue or causing thermal damage. However, an ultraviolet laser system usually is generally rather bulky to be optimal for a dental operation.

As can be seen by the above-described prior art, a relatively thick pool of water on the surface of dental material was previously thought to be ineffective for providing cooling in conjunction with an infrared laser that is highly absorbed by water. Steiner et al. have used laser radiation to induce the expansion of a film of water to provide a mechanical cutting force, whereas Rizoiu et al. have used a laser to energize fine water droplets to provide an alternative, but similar mechanical cutting force.

However, it is found surprisingly that a water layer acts as an effective cooling means. In contrary to the teachings of the prior art, the use of a coolant delivery system that forms a pool of water on the surface of a tooth improved the results of treatment significantly even in conjunction with an infrared radiation at a wavelength in the range of 2 to 5 microns. Furthermore, a relatively thick layer of water provides a greater cooling effect and allows the use of greater laser fluence (e.g., energy level), which improves the ablation process rate.

Certain embodiments of the present invention address the above-mentioned drawbacks of the prior art by providing a method and system that avoids unwanted heating of treatment surface and provides an efficient and high quality cutting by a combination of a wavelength range and a coolant spray. In one example, pulsed laser light is used to provide localized energy deposition for cutting without thermal damage and charring to the surrounding tissue.

Embodiments of the present invention provide an apparatus and method for removing carious regions using laser radiation, for example, infrared laser radiation, and a coolant. A carious region may comprise plaque, dental caries, decay tissue, or lesions. The use of laser radiation enables a more accurate and painless treatment of carious regions than conventional drilling machines. Carious regions are removed from hard surfaces without charring of tissue at the target area by preventing excess heat from being generated. By preventing charring of tissue at the target area, the ablation process can be executed without interruption. Also, the potential for dentine or enamel cracking as well as damage to the nerve system and other pulp structure in the pulp chamber is greatly reduced by preventing excess heat from being generated at the target and surrounding areas. Furthermore, the thickness of the coolant layer on the tooth surface does not need to be critically controlled. Thus, the coolant used in the present invention may remain pooled on the tooth surface during laser irradiation. Moreover, a drying step, as required in previous processes, is eliminated.

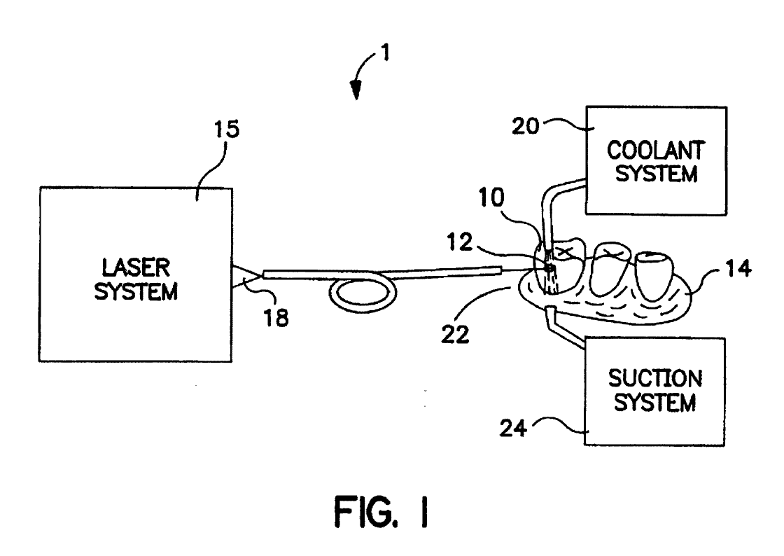

Turning to FIG. 1, an apparatus for removing carious regions, such as plaque and decay tissue, implementing the present invention is shown generally at 1. A tooth 10 having carious region 12 located thereon is shown protruding from a gum 14. A laser system 15 is provided to serve as a source of infrared radiation 18 for the apparatus 1. A coolant delivery system, generally indicated at 20, is provided for delivering coolant to the target area 22. A suction system, indicated at 24, is provided for collecting excess coolant and debris from the target area 22.

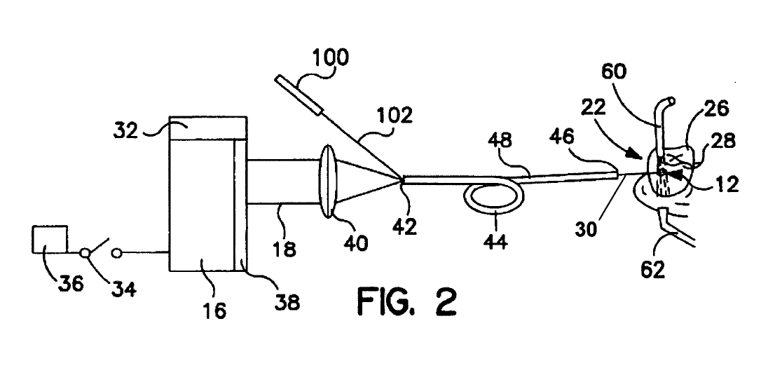

Referring now to FIG. 2, an infrared laser generator 16 is employed to generate pulsed infrared radiation 18 having a wavelength generally in the range of 2 to 5 microns. The infrared laser generator 16 may be set to operate at a predetermined fluence to selectively ablate a single target material since healthy enamel 26, healthy dentin 28, and carious region 12 each have different energy fluence thresholds for ablation, as well as different absorption coefficients which describe the characteristic depths to which infrared radiation 18 is absorbed. The use of the infrared laser generator 16 advantageously permits selective ablating of carious region 12 while leaving healthy dentin 28 and enamel 26 essentially unaffected.

Furthermore, a control panel 32 provides means for adjusting the frequency, energy, and duration of the pulses of radiation 18 generated by the laser 16. An activation switch 34 is coupled between the laser 16 and a power supply 36 to activate the laser 16.

The laser generator 16 in one example produces a pulsed output beam 30 with a pulse repetition rate of from about 1 to about 10,000 pulses per second and laser energy of about 0.01 millijoules per pulse to 5 joules per pulse. In addition, the laser 16 has a wavelength approximately between 2 and 5 microns, which has been shown to be particularly effective in eradicating carious region 12. The diameter of the output beam 30 at the target area 22 in one example is between 0.1 mm to 5 mm.

Although not to be interpreted as limiting, the following lasers have been found to be particularly useful in conjunction with the present invention: Er:YAG laser at approximately 2.94 microns; CTE:YAG laser at approximately 2.69 microns; Er:YALO3 laser having a wavelength in a range of 2.71 to 2.86 microns, CTH:YAG laser at approximately 2.1 microns, and Ho:YAG laser at approximately 2.1 microns.

Optics 38 such as mirrors, lenses or prisms are optionally employed to direct the radiation 18 from the laser 16 to a lens 40. The lens 40 directs and focuses the radiation 18 into one end 42 of a light guide 44. It should be noted that the lens 40 can include more than one element and can be shaped to any number of configurations and focal lengths as desired. Furthermore, an optical output connector (not shown) can be interposed between the laser 16 and the light guide 44 if desired.

The light guide 44, which may be an optical fiber or a mirror and lens system, is provided to direct and transfer the radiation 18 along its interior to an exit end 46. In one embodiment, an optical fiber is used since its flexibility and ease of positioning facilitates operation within the confines of a human mouth.

The exit end 46 in one example is shaped so as to concentrate or focus the output beam 30 exiting therefrom. Alternately, a lens may be attached to the exit end 46 for this purpose. In either case, it is desirable to provide a relatively short focal length at the exit end 46 for most dental applications. The output beam 30 exiting from the exit end 46 is used to ablate carious region 12. It should be noted that the light guide 44 may be incorporated into a hand piece at its free end 48 so as to provide ease of operation for a dentist.

In one embodiment of the present invention, a low power laser 100 operating in the visible spectrum is incorporated into the laser system 15. Helium Neon or diode lasers are suitable for this purpose. A guide beam 102 exiting the low power laser 100 travels through the light guide 44 so as to be substantially coincident with the output beam 30 when projected from the exit end 46. In this way, the output beam 30 can be guided to the target area 22 by observing the position of the guide beam 102.

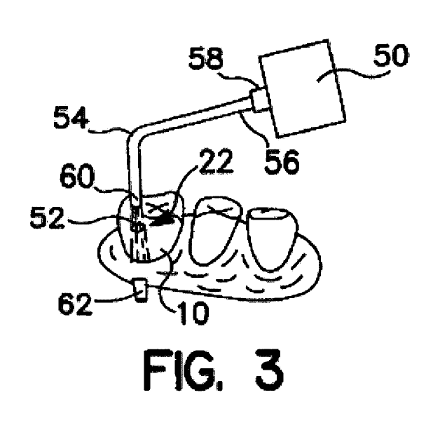

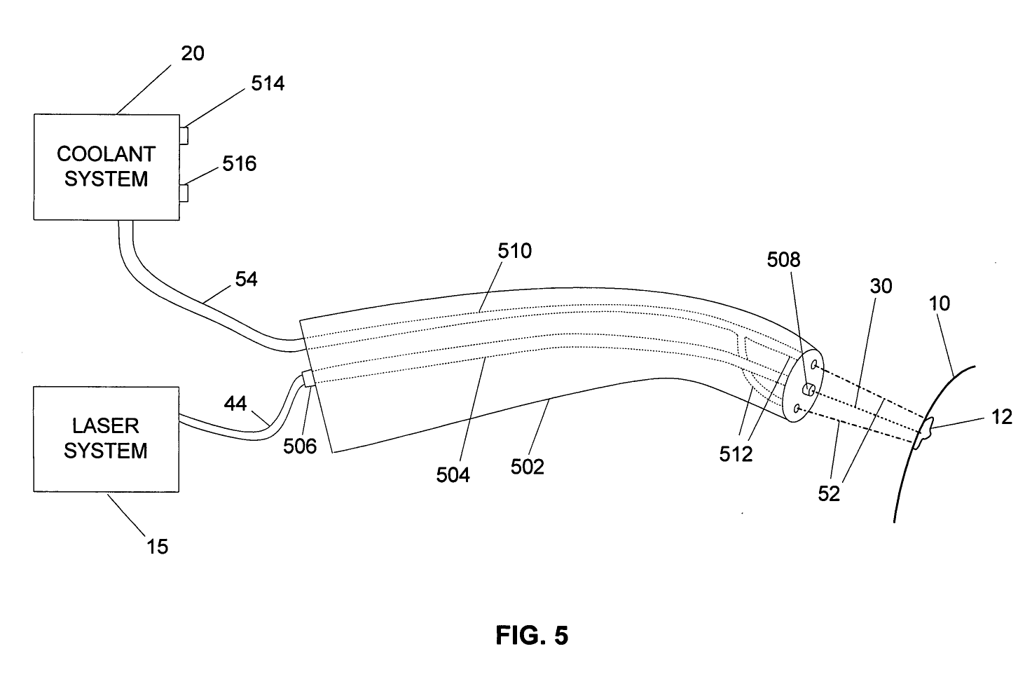

Turning now to FIG. 3, a coolant reservoir 50 is provided for storing coolant to be applied to the tooth 10. In one example, the coolant comprises water 52 cooled to a temperature in the range of 15 degrees Celsius to 30 degrees Celsius. A tube or supply line 54 is coupled at a first end 56 through a connection means 58 to the coolant reservoir 50. The tube 54 is positionable for directing water 52 from an exit end 60 to the target area 22. The water 52 may be delivered intermittently or continuously over the tooth 10. Water delivery systems such as an air/water dental handpiece or other alternatives as found in most dental offices can be used effectively for this purpose. It should be noted that although the coolant in one example comprises water 52 in the form of a spray, other coolants may be used. In one example, the coolant delivery system comprises a gas inlet 514 (FIG. 5) and a coolant inlet 516 (FIG. 5) for pressurized water delivery, for example, via air or nitrogen.

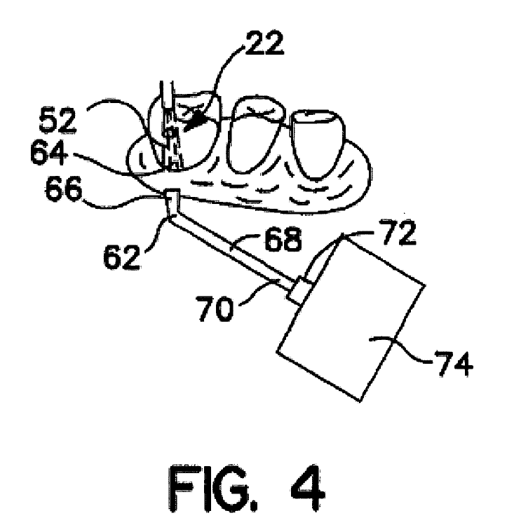

Referring to FIG. 4, a suction tube 62 is provided for removing excess water 52 and debris 64 from the target area 22. The suction tube 62 has a first end 66 adapted for collecting and delivering excess water 52 to an elongated body portion 68. The suction tube 62 also includes an exit end 70 which is coupled by a connection means 72 to a wastep system 74. Suction systems as found in most dental offices are employable for this purpose.

In operation, the low power laser 100 is activated so as to cause the guide beam 102 to exit the light guide 44. Next, the free end 48 of the light guide 44 is maneuvered to aim the exit end 46 generally at a predetermined target area 22 having carious region 12 thereon. Proper aiming is indicated by the guide beam 102 impinging upon the target area 22.

After properly positioning the guide beam 102, the coolant delivery system 20 is operated so as to spray a layer of water 52 on the surface of the tooth 10. The suction system 24 is then operated so as to collect excess water 52. Next, the laser 16 is activated to produce infrared radiation 18. Since the output beam 30 is substantially coincident with the guide beam 102 it impinges upon the target area 22. Finally, the light guide 44 is maneuvered as desired to cause the output beam 30 to impinge upon selected portions of the target area 22.

In accordance with the invention, the output beam 30 passes through the layer of water 52 to eradicate a carious region 12 located beneath the water 52. The water 52 is collected by the suction system 24 during ablation when necessary or continuously to prevent buildup of debris 64 and water 52.

The water 52 may be sprayed continuously so as to pool or form a relatively thick layer on the tooth 10 during laser treatment. Thickness of the water 52 on the tooth 10 in one example is approximately in the range of 0.01 to 3.0 millimeters. It should be noted that the water 52 is much more effective in removing excess heat if it is sprayed on the tooth 10 substantially simultaneously during laser irradiation. In one example, the laser pulse duration, energy level and frequency are set prior to the ablation process. However, these parameters may be adjusted during the procedure if desired. For instance, a quicker rate of ablation may be achieved by increasing the laser energy level. One effective fluence level for removing carious region 12 without causing thermal damage to surrounding areas is approximately 0.07 to 10 J/cm². Effective wavelengths and frequencies are in the range of 2 to 5 microns and 1-1000 Hz respectively.

Turning to FIG. 5, another implementation of the apparatus 1 comprises the laser system 15, the coolant system 20, and a handpiece 502. The handpiece 502 in one example comprises an optical fiber 504 for guiding the output beam 30 towards the tooth 10. One end of the handpiece 502 comprises a fiber connector 506 for connection to the laser system 15. The other end of the handpiece 502 comprises a fiber tip 508. The fiber tip comprises a micro-lens that can focus the output beam 30 into a relative small area for a precision removal of a carious regions on the tooth 10. The fiber tip 508 in one example is removable and can be changed in order to use new or sterilized fiber tips for each patient.

The handpiece 502 comprises a tube 510 for delivery of the water 52 or other coolant. The tube 510 comprises one or more outlets 512, for example, a single outlet or multiple outlets for more uniformly distributed water distribution. A water spray in one example is aimed directly at the dental surface. In another example, the water spray is aimed indirectly at, or in the proximity of, the dental surface. For example, the momentum and/or surface tension of the water can form a layer of water in the area under laser treatment even if it aims away from it. If the water spray is aimed away from the laser treatment area, the amount of water droplets in the air volume in front of the tooth is reduced. The reduced amount of water droplets in front of the tooth reduces attenuation of the laser beam by the water droplets. In another example, the water spray is aimed at a relatively large angle of incidence toward the treatment surface. Thickness of the layer of water can be controlled by changing a flow rate of the water spray. The layer of water in one example is created before starting of the laser treatment to reduce chances for thermal heating of the dental tissue.

Alternative implementations of the handpiece 502 are configured to create a stream or irrigation flow of water instead of the spray. The stream may be gravity fed, pressurized, or any combination thereof. The stream may also be used in combination with one or more sprays of water. Another implementation comprises a first handpiece for the laser delivery system 15 and a second handpiece for the coolant delivery system 20.

One advantage of the current invention is that water spray may be used without critically controlling the thickness of the layer of water as required in the prior art. Furthermore, a continuous flow of water can be utilized thereby alleviating the need to dry the target area between pulses of radiation. Another advantage is that a relatively thick layer of water is highly effective in preventing thermal damage at the target site, surrounding areas and in the pulp chamber since excess heat is prevented from being generated. Charring is also alleviated with water cooling and allows the uninterrupted use of higher laser power which increases the penetration depth per pulse and ablation rate. In addition, an infrared laser is highly effective in selectively ablating carious material while leaving healthy dentine and enamel tissue unaffected.

Those skilled the art can now appreciate from the foregoing description that the broad teachings of the present invention can be implemented in a variety of forms. Therefore, while this invention has been described in connection with particular examples thereof, the true scope of the invention should not be so limited since other modifications will become apparent to those skilled in the art upon a study of the drawings, specification and following claims.

What is claimed is:

1. An apparatus, comprising:
a coolant delivery system that creates a layer of coolant over a target area, wherein the target area comprises at least one carious region; and
a laser generator that directs a laser beam to the target area to ablate the at least one carious region;
wherein the laser generator operates substantially simultaneously with the coolant delivery system to direct the laser beam through the layer of coolant to the target area;
wherein the layer of coolant cools the target area to reduce thermal damage from the laser beam.

2. The apparatus of claim 1, wherein the laser generator comprises an infrared laser generator;
wherein the infrared laser generator creates the laser beam with a wavelength approximately between two and five microns.

3. The apparatus of claim 2, wherein the infrared laser generator comprises one of a Er:YAG laser, a CTE:YAG laser, a Er:YALO3 laser, a CTH:YAG laser, or a Ho:YAG laser.

4. The apparatus of claim 1, wherein the laser beam comprises a pulsed laser beam with a repetition rate approximately between one and 10,000 pulses per second.

5. The apparatus of claim 4, wherein the pulsed laser beam comprises an energy level approximately between 0.01 millijoules per pulse and five joules per pulse.

6. The apparatus of claim 1, wherein the laser generator creates the laser beam with a predetermined fluence level to selectively ablate the at least one carious region with reduced thermal damage to the target area.

7. The apparatus of claim 6, wherein the predetermined fluence level is approximately between 0.05 and 10.0 J/cm².

8. The apparatus of claim 6, wherein the laser generator selects the predetermined fluence level based on an absorption coefficient of the at least one carious region.

9. The apparatus of claim 1, wherein the layer of coolant comprises a thickness approximately between 0.01 millimeters and 3.0 millimeters.

10. The apparatus of claim 1, wherein the coolant delivery system begins delivery of the layer of coolant before a start of the laser beam by the laser generator.

11. The apparatus of claim 1, wherein the coolant delivery system is configured to provide a continuous flow, irrigation, stream, or pool of the coolant over the target area.

12. The apparatus of claim 1, wherein the coolant delivery system comprises an indirect aim of a spray of the coolant at the target area to reduce attenuation of the laser beam through the spray of the coolant.

13. The apparatus of claim 12, wherein the indirect aim of the spray of the coolant, a momentum of the spray of the coolant, and a surface tension of the coolant cooperate to form the layer of coolant over the target area.

14. The apparatus of claim 1, wherein the coolant comprises water.

15. An apparatus, comprising:
a coolant delivery system;
a laser generator; and
a handpiece coupled with the coolant delivery system to receive a supply of coolant, and configured to create a layer of coolant over a target area, wherein the target area comprises at least one carious region;
wherein the handpiece comprises an optical fiber that is optically coupled with the laser generator to receive a laser beam from the laser generator and direct the laser beam through the layer of coolant to the target area to ablate the at least one carious region;
wherein the handpiece substantially simultaneously creates the layer of coolant over the target area and directs the laser beam through the layer of coolant to the target area to ablate the at least one carious region;
wherein the layer of coolant cools the target area to reduce thermal damage from the laser beam.

16. The apparatus of claim 14, wherein the laser generator comprises an infrared laser generator;
wherein the infrared laser generator creates the laser beam with a predetermined fluence level approximately between 0.05 and 10.0 J/cm².

17. The apparatus of claim 14, wherein the handpiece is configured to create the layer of coolant with a thickness approximately between 0.01 millimeters and 3.0 millimeters.

18. The apparatus of claim 16, wherein the handpiece is configured to create the layer of coolant as a continuous flow of coolant over the target area during operation of the laser beam.

19. The apparatus of claim 14, further comprising:
a suction system that removes excess coolant from the target area.

20. The apparatus of claim 1, wherein the coolant comprises water.

21. A method, comprising the steps of:
creating a pool of coolant over a target area for cooling of the target area, wherein the target area comprises at least one carious region; and
substantially simultaneously directing an infrared laser beam through the pool of coolant to the at least one carious region to ablate the at least one carious region.

22. The method of claim 19, wherein the step of creating the pool of coolant comprises the step of:
indirectly aiming a spray of the coolant at the target area to reduce attenuation of the infrared laser beam through the spray of the coolant.

23. An apparatus, comprising:
means for creating a pool of coolant over a target area for cooling of the target area, wherein the target area comprises at least one carious region; and
means for substantially simultaneously directing an infrared laser beam through the pool of coolant to the at least one carious region to ablate the at least one carious region.

24. The apparatus of claim 23, wherein the means for creating the pool of coolant comprises:
means for indirectly aiming a spray of the coolant at the target area to reduce attenuation of the infrared laser beam through the spray of the coolant.
